(19) United States
(12) Patent Application Publication
(10) Pub. No.: US 2026/0021039 A1
(43) Pub. Date: Jan. 22, 2026

(54) VISCOELASTIC COMPOSITION FOR OPHTHALMIC SURGERY

(71) Applicant: Iromed Group S.r.l., Roma RM (IT)

(72) Inventor: Ciro Caruso, Napoli NA (IT)

(73) Assignee: Iromed Group S.r.l., Roma RM (IT)

(21) Appl. No.: 18/775,086

(22) Filed: Jul. 17, 2024

(51) Int. Cl. A61K 9/00; A61K 49/00; A61P 27/02; A61P 41/00

(52) U.S. Cl. CPC A61K 9/0048; A61K 49/006; A61K 49/0071; A61P 27/02; A61P 41/00

(57) ABSTRACT

The present invention relates to a viscoelastic composition for use in an ophthalmic surgery method, which is transparent and at the same time colored, which allow the surgeon to easily identify the composition during the ophthalmic surgery which facilitates viscoelastic removal at the end of the operation. The present invention also concerns an ophthalmic viscoelastic device which includes the viscoelastic composition of the present invention, and a method of performing an ophthalmic surgery on the eye of a subject, having a step of administering to said eye an effective amount of the viscoelastic composition.

### TECHNICAL FIELD OF THE INVENTION

The present invention relates to a viscoelastic composition for use in an ophthalmic surgery method, which is transparent and at the same time colored, which allow the surgeon to easily identify the composition during the ophthalmic surgery which facilitates viscoelastic removal at the end of the operation. The present invention also concerns an ophthalmic viscoelastic device comprising the viscoelastic composition of the present invention, and a method of performing an ophthalmic surgery on the eye of a subject, comprising a step of administering to said eye an effective amount of the viscoelastic composition.

### BACKGROUND OF THE INVENTION

Ophthalmic Viscosurgical (or viscoelastic) Devices (OVD) are non-active, transparent (those currently known in the art) and gelatinous compositions, with viscous and elastic properties, used in ophthalmic surgery for the execution of mechanical surgical maneuvers in order to create and maintain spaces necessary during the surgical maneuvers. In particular, OVD are used to maintain adequate anterior chamber depth, to prevent mechanical damage to ocular tissues, and to act as buffering agents or vitreous substitutes.

The typical individual characteristics of the various compositions on the market are the result of the length of the chains and the intra- and intermolecular interactions of the compounds constituting the viscoelastic substance. All viscoelastic materials have the potential to produce a dramatic increase in intraocular pressure if they are not adequately removed from the anterior chamber after surgery. The mechanism of action is essentially mechanical. It is important to take into account the viscoelasticity of the preparation in relation to the type of operation to be performed. No pharmacological, metabolic or immunological mechanism is involved in the action of the OVD.

Currently all viscoelastic compositions used in ophthalmic surgery available on the market are transparent. Due to their transparency, it is difficult for the surgeon to identify the viscoelastic, which represents a problem of considerable importance since, once the operation is finished, the viscoelastic must be completely removed; leaving residues can cause increased eye pressure, resulting in serious eye problems.

### SUMMARY OF THE INVENTION

In order to solve the problems of the prior art, the Authors of the present invention have developed a viscoelastic composition for use in a method of treatment of ophthalmic surgery, which maintains an adequate depth of the anterior chamber to prevent mechanical damage to the ocular tissues and is colored, which facilitates viscoelastic removal at the end of the operation.

The viscoelastic composition developed by the authors of the present invention is advantageously indicated for:
Cataract operations with or without intraocular lens implantation;
Glaucoma surgery;
Corneal surgery;
Surgical interventions for retinal detachment.

Thanks to its visibility, the viscoelastic composition of the present invention makes the removal phase of the viscoelastic extremely easy and allows the surgeon to visually explore all the spaces without the risk of leaving viscoelastic residues inside the operated bulb, which are dangerous for the hypertonic damage.

Furthermore, the physic-chemical characteristics of the composition for use of the present invention allow the viscoelastic to guarantee the absorption of artificial wavelengths (such as UV and blue light between 400-450 nm), produced by the light of the surgical ocular microscope. This prevents these radiations from causing photooxidative damage to the macula during surgical maneuvers. It also acts as a deactivator, neutralizing free radicals formed during cataract surgery through the use of ultrasound. The viscoelastic composition of the present invention remains, during cataract surgery, within the eye, thereby creating and maintaining a deep anterior chamber and a clear field of vision during surgery and allows for safe and controlled manipulation at interior of the eye, minimizing any trauma to the corneal endothelium and other tissue organs, and is also used to maneuver, control and separate tissues in an atraumatic way, protecting them from mechanical impacts.

Therefore, objects of the present invention include:
a viscoelastic composition for use in an ophthalmic surgery method comprising a viscoelastic polymer and a dye, wherein said viscoelastic composition is transparent and colored,
an ophthalmic viscoelastic device comprising the viscoelastic composition for use according to the present invention, wherein said viscoelastic composition is in a syringe, and
a method of performing an ophthalmic surgery on the eye of a subject, comprising a step of administering to said eye an effective amount of the viscoelastic composition according to the present invention.

### Glossary

Unless otherwise defined herein, scientific, and technical terms used in connection with the present invention shall have the meanings that are commonly understood by those of ordinary skill in the art. Further, unless otherwise required by context, singular terms shall include pluralities and plural terms shall include the singular.

In any point of the present specification or of the claims, the expression “comprising” or “comprise(s)” can be replaced by “consisting of” or “consist(s) of”.

### DETAILED DESCRIPTION OF THE INVENTION

The present invention relates to a viscoelastic composition for use in an ophthalmic surgery method comprising a viscoelastic polymer and a dye, wherein said viscoelastic composition is transparent and colored. The terms “transparent and colored” refer to specific desirable properties of the viscoelastic composition for use according to the present invention. Being transparent, the viscoelastic composition must be clear and allow light to pass through without significant scattering or absorption. This transparency is crucial during ophthalmic surgery to ensure that the surgeon has an unobstructed view of the surgical field, including the structures within the eye. Clear viscoelastic materials help maintain visibility, which is essential for precision and safety during delicate procedures like cataract removal and lens implantation. The viscoelastic composition for use according to the present invention is intentionally colored by addition of the dye, and according to a preferred embodiment of the present invention the color is yellow. The purpose of coloring is to enhance the surgeon's ability to see the material within the eye. A slight tint can help the surgeon differentiate the viscoelastic material from the eye's natural tissues and other substances used during surgery. This can be especially useful in ensuring that the material is completely removed from the eye at the end of the procedure to avoid post-operative complications. Furthermore, the color provided by the dye allows the viscoelastic to guarantee the absorption of artificial wavelengths (such as UV and blue light between 400-450 nm), produced by the light of the surgical ocular microscope. This prevents these radiations from causing photooxidative damage to the macula during surgical maneuvers. It also acts as a deactivator, neutralizing free radicals formed during cataract surgery through the use of ultrasound. In summary, a viscoelastic composition for use in ophthalmic surgery being “transparent and colored” implies that the material should be mostly transparent to allow clear visibility while also potentially having a slight tint to aid the surgeon in visualizing the material during the procedure.

In one embodiment, the dye in the viscoelastic composition comprises riboflavin. As well known by the person skilled in the art, Riboflavin, also known as vitamin B2, is a water-soluble vitamin.

In one embodiment, said viscoelastic polymer is hyaluronic acid or sodium hyaluronate and/or a mixture thereof.

Hyaluronic acid is a naturally occurring polysaccharide found extensively in the extracellular matrix of connective tissues. It is a glycosaminoglycan with a high molecular weight, notable for its ability to retain water, providing significant hydration and lubrication. Hyaluronic acid is clear, viscous, and biocompatible, making it essential for maintaining the structural integrity and functional performance of tissues without eliciting an immune response.

Sodium hyaluronate is a specific fraction of sodium hyaluronate widely distributed in the extracellular matrix of connective tissue. It is a compound with a high molecular weight, high viscosity, transparent and without antigenicity.

In one embodiment, the dye consists of riboflavin conjugated to a second viscoelastic polymer. Preferably, said second viscoelastic polymer is hyaluronic acid or sodium hyaluronate and hyaluronic acid or sodium hyaluronate.

In one preferred embodiment, the second viscoelastic polymer is hyaluronic acid or sodium hyaluronate and hyaluronic acid or sodium hyaluronate and is conjugated to riboflavin. Hyaluronic acid or sodium hyaluronate conjugated with a riboflavin is disclosed in European patent EP3419590B1 herein incorporated by reference. It is a biopolymer belonging to the class of glycosaminoglycans with high molecular weight. Conjugated hyaluronic acid or sodium hyaluronate are biological and biocompatible substances. According to EP3419590B1, the conjugation between hyaluronic acid or sodium hyaluronate and riboflavin is by a covalent bond, in particular is an ester bond between the carboxylic group of hyaluronic acid and the C20 carbon of riboflavin according to the structure formula (I):

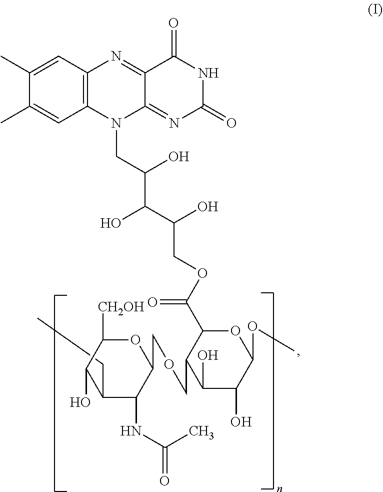

Moreover, the molecular weight of said hyaluronic acid or sodium hyaluronate conjugated to riboflavin is comprised between 200.000 and 5.000.000 of Dalton, in particular between 200.000 and 800.000 Dalton, wherein said molecular weight is the average molecular weight of hyaluronic acid without considering the contribution of the weight of riboflavin.

The degree of substitution of riboflavin in the esters in the hyaluronic acid or sodium hyaluronate conjugated to riboflavin is between 2 and 7%.

The composition contains hyaluronic acid or sodium hyaluronate conjugated to riboflavin or vitamin B2, has a characteristic transparent straw yellow color that improves contrast. Thus, it ensures excellent visualization and safe capsulorhexis.

Hyaluronic acid or sodium hyaluronate conjugated with a riboflavin boasts the benefits of both. In particular, the light-screening action of Riboflavin allows the disappearance or reduction of photophobia observed as early as two hours after instillation of the eye drop, while the longer residence time in the tear film allows the patients to reduce the frequency of instillation while using the hyaluronic acid or sodium hyaluronate conjugated with a riboflavin. The hyaluronic acid or sodium hyaluronate creates a stable protective layer over the tear film, counteracting the premature evaporation of the tear itself, it lubricates the cornea and conjunctiva, and it has moisturizing properties. The Riboflavin on the other hand gives absorbing power on UV radiation and on the spectrum of harmful blue light.

In one embodiment the viscoelastic polymer has a molecular weight from 200.000 to 5.000.000 Da, preferably from 200.000 to 800.000 Da.

In one embodiment the viscoelastic polymer is present in an amount from 1.2 to 1.4% w/W.

In one embodiment the dye comprising riboflavin is present in an amount from 0.3 to 0.5% w/w.

In one embodiment, the viscoelastic composition for use according to the present invention further comprises at least one solvent and at least one excipient. The person skilled in the art know which solvents and excipient are suitable for the intended use of ophthalmic surgery. Preferably, the solvent is selected from water and said excipient is a buffering agent, an isotonic agent or mixtures thereof. In one preferred embodiment, the buffering agent is selected from monobasic sodium phosphate or dibasic sodium phosphate, and the isotonic agent is selected from sodium chloride

In one embodiment, the buffering agent is present in an amount from 0.2 to 1.4% w/w, preferably from 1.20 to 1.35% w/w, and/or the isotonic agent is present in an amount from 0.60 to 0.90% w/w.

In one embodiment, the viscoelastic composition for use according to the present invention has a pH from 6.8 to 7.6.

In one embodiment, the viscoelastic composition for use according to the present invention has an osmolality from 200 to 400 mOsm/Kg.

In one embodiment, the viscoelastic composition for use according to the present invention has a dynamic viscosity from 60000 to 250000 mPa.

In one embodiment, the viscoelastic composition for use according to the present invention has a color and transparency.

In one preferred embodiment, the viscoelastic composition for use according to the present invention comprises sodium hyaluronate in an amount ranging from 0.5 to 3%, preferably from 1 to −2.5%, still more preferably from 1.2 to 1.9% w/w and hyaluronic acid or sodium hyaluronate conjugated to riboflavin in an amount ranging from 0.05 to 1%, preferably from 0.1 to 0.8%, still more preferably from 0.3 to 0.5% w/w.

In one embodiment, the viscoelastic composition for use according to the present invention comprises sodium hyaluronate, and hyaluronic acid or sodium hyaluronate conjugated to riboflavin in an amount ranging from:

| | Composition | |
|---|---|---|
| Substance | % p/p | Function |
| HYALURONIC ACID | 0.3-0.5 | FUNCTIONAL |
| OR SODIUM | | INGREDIENT |
| HYALURONATE | | |
| CONJUGATED | | |
| WITH RIBOFLAVIN | | |
| SODIUM | 1.2-1.9 * | THICKENING |
| HYALURONATE | | |

* Quantities refers to an away of Sodium Hyaluronate equal to 100%

In one embodiment the ophthalmic surgery method for which the viscoelastic composition herein disclosed is used is a cataract surgery with or without intraocular lens implantation, Glaucoma surgery, Corneal surgery, Surgical interventions for retinal detachment.

In the ophthalmic surgery, the composition for use according to the invention can be used for filling up the vitreous body as well as for stabilizing the anterior chamber and for protection of the highly sensible endothelial cell layer of the cornea during surgery on the anterior eye portions, especially the surgery of the cataract.

During the phacoemulsification, the anterior chamber of the eye is filled with the ophthalmologic composition comprising the coloured viscoelastic solution. The viscoelastic composition for use according to the present invention is used as a surgical aid to protect intraocular tissues, for example the corneal endothelium, during the phacoemulsification, as a space maintainer to maintain the anterior chamber of the eye, and to facilitate intraocular maneuvers, for example to make a controlled capsulorhexis. In the preferred embodiment wherein the composition comprises sodium hyaluronate or hyaluronic acid and riboflavin conjugated with a second viscoelastic polymer (i.e. sodium hyaluronate or hyaluronic acid), as the dye is covalently bound to the second viscoelastic polymer, the dye do not diffuse out of the solution and therefore do not negatively affect or stain the surrounding tissues.

One object of the present invention is also an ophthalmic viscoelastic device comprising the viscoelastic composition for use according to the present invention, wherein said viscoelastic composition is in a syringe. The viscoelastic composition for use according to the present invention, after sterilization can be furnished as a medical device, allocated inside a class syringe, such as a 25 ml syringe with polycarbonate luer lock. The syringe can be closed with an elastomeric rubber luer tip and an external polypropylene cap and a rubber stopper. The syringe can be equipped with a polypropylene piston and polypropylene back stop. The syringe is sterile and can be designed for single use. The syringes, preassembled with rubber luer tip and external polypropylene cap, can be packaged in a plastic nest.

Further object of the present invention is a method of performing an ophthalmic surgery on the eye of a subject, comprising a step of administering to said eye an effective amount of the viscoelastic composition according to any embodiments herein disclosed of the present invention.

In one embodiment, the method of the present invention comprises a step of filling up an anterior chamber of said eye of the patient.

In one embodiment, the method of the present invention comprises a further step of removing said viscoelastic composition from said eye of the subject. Thanks to its colouring and hence visibility, the composition of the present invention, as discussed above, allows the surgeon to better visualize the composition and to remove in a more efficient way.

In one embodiment, said ophthalmic surgery is a cataract surgery with or without intraocular lens implantation, Glaucoma surgery, Corneal surgery, Surgical interventions for retinal detachment.

### EXAMPLES

During the development phase of the colored ophthalmic viscoelastic, particular attention was paid to obtaining a color that ensured both the transparency necessary for effective and safe use in surgical procedures and optimal visibility for surgeons.

The development team's main goal was to create a viscoelastic solution that could be easily visualized during surgery without compromising its transparent and viscoelastic properties. This balance is critical to improving the precision and safety of ophthalmic procedures. In the most preferred embodiment, the choice of riboflavin (vitamin B2) as a coloring agent was strategic and based on numerous tests and analyses. Riboflavin was selected for the following reasons:
Biological Safety: Riboflavin is a biocompatible and safe component for intraocular use, as demonstrated by scientific literature and our internal tests.
Visual Effectiveness: The yellow color obtained with riboflavin provides sufficient contrast to improve the visibility of the viscoelastic within the operating field, without being too intense to obscure the surgeon's vision.
Maintenance of Transparency: Despite the coloration, riboflavin maintains the transparency of the viscoelastic, allowing clear visualization of the intraocular structures and facilitating surgical maneuvers.

During the development of the colored viscoelastic, numerous tests were conducted to ensure that the solution met the highest quality and safety standards:
Transparency Tests: Optical tests were carried out to ensure that the solution maintained a high degree of transparency, essential for viewing ocular structures during surgery.
Visibility Test: The color has been calibrated to ensure optimal visibility under different lighting conditions, typical of operating theatres.
Stability Test: The stability of the color has been verified through long-term stability studies to ensure that the viscoelastic maintains its properties throughout the shelf life.

The results obtained confirm that the colored viscoelastic offers an ideal combination of transparency and visibility, significantly improving the surgical experience and increasing the safety of the procedures. The yellow coloration does not interfere with visual clarity but, instead, provides useful contrast that helps surgeons precisely monitor and control viscoelastic application.

The combination of transparency and visibility obtained represents a significant technological advancement that will improve surgical outcomes and patient safety.

### Example of Composition #1

| | Composition | |
|---|---|---|
| Substance | % p/p | Function |
| HYALURONIC ACID | 0.3-0.5 | FUNCTIONAL |
| CONJUGATED | | INGREDIENT |
| WITH RIBOFLAVIN | | |
| SODIUM | 1.2-1.4 * | THICKENING |
| HYALURONATE | | |
| SODIUM | 0.2-0.22 | BUFFERING AGENT |
| DIHYDROGEN | | |
| PHOSPHATE | | |
| DIHYDRATE | | |
| DISODIUM | 1.00-1.10 | BUFFERING AGENT |
| HYDROGEN | | |
| PHOSPHATE | | |
| DODECAHYDRATE | | |
| SODIUM | 0.60-0.90 | ISOTONIC AGENT |
| CHLORIDE | | |
| WATER FOR | ENOUGH TO | SOLVENT |
| INJECTION | 100 | |

* Quantities refers to an away of Sodium Hyaluronate equal to 100%

### Example of Composition #2

| | Composition | |
|---|---|---|
| Substance | % p/p | Function |
| HYALURONIC ACID | 0.3-0.5 | FUNCTIONAL |
| CONJUGATED | | INGREDIENT |
| WITH RIBOFLAVIN | | |
| SODIUM | 1.7-1.9* | THICKENING |
| HYALURONATE | | |
| SODIUM | 0.20-0.22 | BUFFERING AGENT |
| DIHYDROGEN | | |
| PHOSPHATE | | |
| DIHYDRATE | | |
| DISODIUM | 1.00-1.10 | BUFFERING AGENT |
| HYDROGEN | | |
| PHOSPHATE | | |
| DODECAHYDRATE | | |
| SODIUM | 0.60-0.90 | ISOTONIC AGENT |
| CHLORIDE | | |
| WATER FOR | ENOUGH TO | SOLVENT |
| INJECTION | 100 | |

*Quantities refers to an away of Sodium Hyaluronate equal to 100%

### Chemical-Physical Properties of Example Composition #1

| | CONTROLS | SPECIFIC | |
|---|---|---|---|
| CHEMICAL- | Solution appearance | Clear, transparent with slight | |
| PHYSICAL | | yellow reflections | |
| | pH | 6.8-7.6 | |
| | osmolality | 200-400 | mOsm/kg |
| | Dinamic viscosity | 60′000-120′000 | mPa · s |

### Chemical-Physical Properties of Example Composition #2

| | CONTROLS | SPECIFIC | |
|---|---|---|---|
| CHEMICAL- | Solution appearance | Clear, transparent with slight | |
| PHYSICAL | | yellow reflections | |
| | pH | 6.8-7.6 | |
| | osmolality | 200-400 | mOsm/kg |
| | Dinamic viscosity | 130′000-250′000 | mPa · s |

1. A viscoelastic composition for use in an ophthalmic surgery method, comprising a viscoelastic polymer and a dye, wherein said viscoelastic composition is transparent and colored.

2. The viscoelastic composition according to claim 1, wherein said dye comprises riboflavin.

3. The viscoelastic composition according to claim 1, wherein said viscoelastic polymer is hyaluronic acid or sodium hyaluronate and/or a mixture thereof.

4. The viscoelastic composition according to claim 1, wherein said dye consists of riboflavin conjugated to a second viscoelastic polymer.

5. The viscoelastic composition according to claim 4, wherein said second viscoelastic polymer is hyaluronic acid or sodium hyaluronate and hyaluronic acid or sodium hyaluronate and is conjugated to riboflavin.

6. The viscoelastic composition according to claim 1, wherein said viscoelastic polymer and said dye are in a viscoelastic polymer/dye weight ratio from 2:1 to 4:1.

7. The viscoelastic composition according to claim 1, wherein said viscoelastic polymer is present in an amount from 1.2 to 1.4% w/w and/or wherein said dye comprising riboflavin is present in an amount from 0.3 to 0.5% w/w.

8. The viscoelastic composition according to claim 1, having at least one of the following features: a pH from 6.8 to 7.6, and/or an osmolality from 200 to 400 mOsm/Kg and/or a dynamic viscosity from 60000 to 250000 mPa-s.

9. The viscoelastic composition according to claim 1, comprising sodium hyaluronate in an amount from 0.5 to 3% w/w, and hyaluronic acid or sodium hyaluronate conjugated to riboflavin in an amount from 0.05 to 1% w/w.

10. The viscoelastic composition according to claim 1, wherein said ophthalmic surgery method is a cataract surgery with or without intraocular lens implantation, glaucoma surgery, corneal surgery, or surgical interventions for retinal detachment.

11. An ophthalmic viscoelastic device comprising the viscoelastic composition according to claim 1, wherein said viscoelastic composition is in a syringe.

12. A method of performing an ophthalmic surgery on the eye of a subject, comprising a step of administering to said eye an effective amount of the viscoelastic composition according to claim 1.

13. The method according to claim 12, further comprising a step of filling up an anterior chamber of said eye of the patient.

14. The method according to claim 12, comprising a further step of removing said viscoelastic composition from said eye of the subject.

15. The method according to claim 12, wherein said ophthalmic surgery is a cataract surgery with or without intraocular lens implantation, glaucoma surgery, corneal surgery, or surgical interventions for retinal detachment.
